(19) United States
(12) Patent Application Publication
(10) Pub. No.: US 2019/0289874 A1
(43) Pub. Date: Sep. 26, 2019

(54) STAIN PREVENTION FORMULATIONS

(71) Applicant: WM. WRIGLEY JR. COMPANY, Chicago, IL (US)

(72) Inventors: Minmin TIAN, Chicago, IL (US); Michael DODDS, Chicago, IL (US); Eric DOWD, Chicago, IL (US); Holly KNUTSEN, Chicago, IL (US); Amy MO, Chicago, IL (US); Lilian RAMIREZ, Chicago, IL (US)

(21) Appl. No.: 16/302,756

(22) PCT Filed: May 26, 2017
(86) PCT No.: PCT/US17/34705

§ 371 Date: Nov. 19, 2018

Related U.S. Application Data
(60) Provisional application No. 62/343,624, filed on May 31, 2016.

(51) Int. Cl. A23G 4/06; A23G 4/20; A23G 3/54; A23G 3/36; A61K 8/24; A61K 8/42; A61K 8/43; A61K 8/49; A61K 8/11; A61Q 11/00

(52) U.S. Cl. CPC A23G 4/064; A23G 4/20; A23G 3/54; A23G 3/362; A61Q 11/00; A61K 8/42; A61K 8/43; A61K 8/494; A61K 8/11; A61K 8/24

(57) ABSTRACT

Stain prevention oral products comprising one or more stain prevention compounds are provided. The oral products provide stain prevention and stain reduction benefits.

### FIELD

The presently disclosed subject matter relates to stain prevention formulations, particularly for use in oral products. Specifically, the present disclosure is directed to stain prevention formulations for use in chewing gums, chewy candies and compressed mints.

### BACKGROUND

The majority of the people worldwide consider clean, white teeth to be aesthetically desirable. Teeth blemished with extrinsic stains are objectionable both on the basis of cosmetic appearance and also socially as an indication of poor oral hygiene. Most people will form some unsightly extrinsic stains on their teeth over time because the acquired pellicle, which coats the teeth, has a natural tendency to stain. This staining process is promoted by the ingestion of chromogenic foods and beverages such as coffee, tea, or red wine, the use of tobacco products and exposure to certain cationic substances such as tin, iron, and chlorhexidine. Currently, the most widely practiced method for the control of extrinsic stains is daily tooth brushing with dentifrices. However, brushing alone is not enough to completely prevent stain formation. Many people still periodically need to have their teeth professionally whitened.

Current approaches for removal of staining require treatments including high levels of bleaching agents such as hydrogen peroxide, carbamide peroxide, sodium percarbonate, calcium peroxide, etc. However, these compounds are toxic to hard and soft oral tissue. They can potentially damage tooth enamel and cause hypersensitivity. In addition, products containing hydrogen peroxide-based bleaching agents are usually associated with poor stability and product shelf-life. The products also have a bad sensory taste that is hard to mask. The presently disclosed subject matter addresses this need as discussed in detail below.

### SUMMARY OF THE INVENTION

The presently disclosed subject matter is directed to oral compositions comprising at least one stain prevention compound selected from the group consisting of surface modifying agents, hydrogen-bond disrupting agents and combinations thereof. The oral compositions can be a chewing gum, chewy candy or compressed mint.

In certain embodiments, the composition comprises at least two stain prevention compounds.

In other embodiments, the at least one surface modifying agent is selected from the group consisting of lauroyl arginine ethyl ether (LAE), sodium tripolyphosphate (STPP or STP), sodium pyrophosphate (SPP), sodium metatriphosphate (SMTP), and combinations thereof.

In further embodiments, the at least one hydrogen-bond disrupting agent is selected from the group consisting of urea, arginine, guanidine, thiourea, allantoin, and combinations thereof.

In certain embodiments, the gum comprises one surface modifying agent and one hydrogen-bond disrupting agent.

The foregoing has outlined broadly the features and technical advantages of the present disclosure in order that the detailed description that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### BRIEF DESCRIPTION OF THE DRAWINGS

FIGS. 1a-1c display dose responses of various stain prevention formulations versus the change in visual whitening index (y-axis) by bovine teeth test. FIG. 1a includes SPP and STP formulations as compared to a negative control and hydrogen peroxide. FIG. 1b includes urea formulations as compared to a negative control and hydrogen peroxide. FIG. 1c includes arginine and LAE formulations as compared to a negative control and hydrogen peroxide.

FIG. 2a-2c display the effect of combinations of STP-urea, STP-LAE, SPP-urea and SPP-LAE on stain-prevention by bovine teeth test, measured by the change in visual whitening index (y-axis). FIG. 2a displays the effect of a combination of STP-urea at various doses. FIG. 2b displays the effect of combinations of STP-LAE at various doses. FIG. 2c displays the effect of the combinations of SPP-urea and SPP-LAE.

FIG. 3 displays the release of LAE from tab, pellet and stick gum measured by HPLC (y-axis) over time (x-axis).

FIG. 4 depicts the ΔE (y-axis) for each of four tests in the chewing gum machine at t=0, 7 and 14 days.

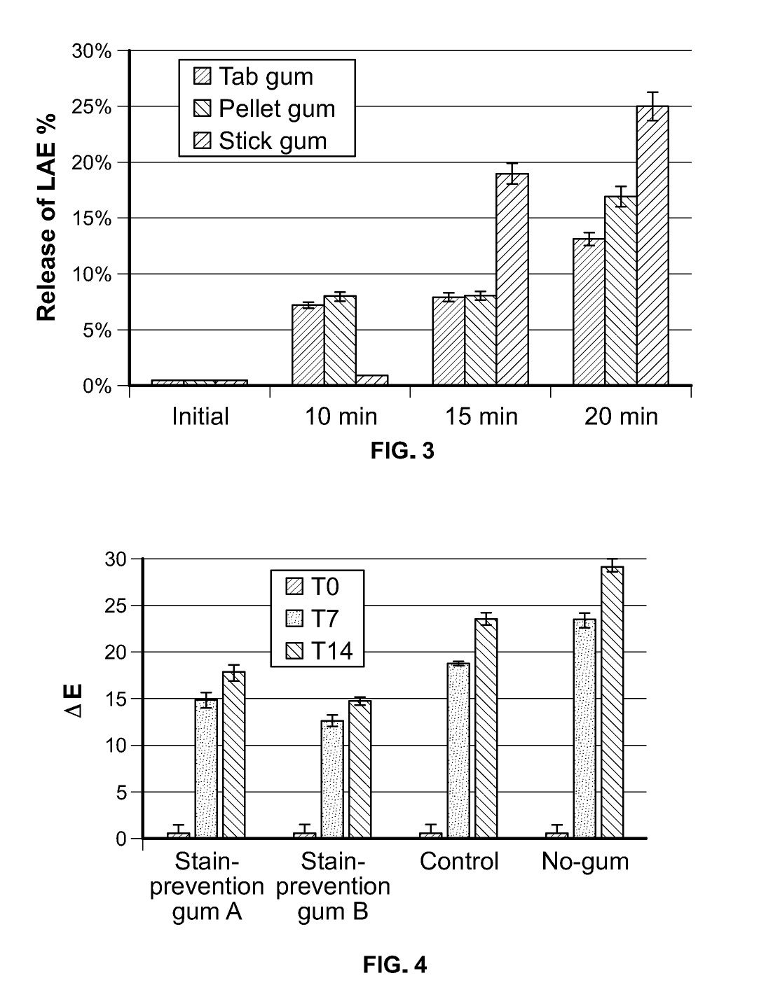

### DETAILED DESCRIPTION

As noted above, there remains a need in the art for stain prevention chewing gum formulations and methods for improving oral health. The presently disclosed subject matter addresses this need through the use of one or more stain prevention compounds that provide stain prevention features sufficient to prevent and/or reduce tooth staining.

### 1. Definitions

The terms used in this specification generally have their ordinary meanings in the art, within the context of this disclosed subject matter and in the specific context where each term is used. Certain terms are discussed below, or elsewhere in the specification, to provide additional guidance to the practitioner in describing the compositions and methods of the disclosed subject matter and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Unless otherwise specified, all percentages used herein are weight percents.

As used herein “admixing,” refers to the process where the stain prevention formulation is mixed with or added to the completed product or mixed with some or all of the components of the product during product formation or some combination of these steps. When used in the context of admixing, the term “product” refers to the product or any of its components. This admixing step can include a process selected from the step of adding the stain prevention formulation to the product, spraying the stain prevention formulation on the product, coating the stain prevention formulation on the product, suspending the stain prevention formulation in the product, painting the stain prevention formulation on the product, pasting the stain prevention formulation on the product, encapsulating the product with the stain prevention formulation, mixing the stain prevention formulation with the product and any combination thereof. The stain prevention formulation can be a liquid, dry powder, spray, paste, suspension and any combination thereof.

As used herein, the term “chewing gum” refers to a flavored substance intended for chewing. The term as used herein also includes bubble gum and confectionery products containing chewing gum. In certain embodiments, chewing gum forms include, but are not limited to, tablets, sticks, solid balls, hollow balls, cut and wrap, and pellets or pillows. Unless otherwise specified, all percentages used herein are weight percents. As used herein, chewing gum contains a water insoluble base portion and a water-soluble bulk portion.

The term “chewy candy” as used herein can have a chewy consistency approximating that of chewing gum, and can be swallowed after mastication.

As used herein, the terms “compressed mint” refers to a hard confectionery product that requires sufficient strength to bite and/or shatter and primarily dissolves in the mouth by sucking.

As used herein, the term “breath-freshening agent” means compounds or compositions that counteract malodor. Breath-freshening agents include, but are not limited to, salts of zinc, salts of copper, polyphenols, mushroom extracts and mixtures thereof.

As used herein, the term “chromogen” means a compound or substance that converts into a colored compound.

As used herein, the term “pellicle” indicates a thin layer of proteins that coats the surface or enamel of a tooth.

As used interchangeably herein, the terms “color” and “color characteristics” refer to the color properties such as hue, chroma, purity, saturation, intensity, vividness, value, lightness, brightness and darkness, and color model system parameters used to describe these properties, such as Commission Internationale de l'Eclairage CIE 1976 CIELAB color space L*a*b* values and CIELCH color space L*C*h° values. In certain embodiments, the whiteness of the teeth tested in the present disclosure can be analyzed with a colorimeter or spectrophotometer, and CIELAB L*a*b* and CIELCH L*C*h° values can be calculated from the spectral data. The L*a*b* and L*C*h° values provide a means of representing color characteristics and assessing the magnitude of difference in whiteness before and after chew.

### 2. Stain Prevention Compounds

The present application relates to stain prevention formulations that include at least one, two, three or more stain prevention compounds. Stain prevention compounds include, but are not limited to, surface modifying agents and hydrogen-bond disrupting agents.

Surface modifying agents prevent staining molecules, e.g., chromogenic foods and beverages, from attaching to a tooth surface.

In certain embodiments, surface modifying agents can include, but are not limited to, lauroyl arginine ethyl ether (LAE), sodium tripolyphosphate (STPP or STP), sodium pyrophosphate (SPP), sodium metatriphosphate (SMTP), and combinations thereof.

SPP, SMTP and STP can also be chelating agents. As chelating agents, they bind bivalent metal ions such as calcium, iron and copper salts and form metal complex. In certain embodiments, they are also tartar controlling agents.

LAE is an amino acid-derived, cationically charged surfactant. In certain embodiments, LAE has a strong germ-kill and biofilm inhibition effect.

In certain embodiments, SPP, SMTP, STP and LAE have a strong affinity for a tooth surface because of structural similarity to hydroxyapatite (or cationic charge in the case of LAE).

Hydrogen-bond disrupting agents break down chromogen and or staining pellicles. In certain embodiments, these agents form strong hydrogen bonds in aqueous solution and disrupt the interaction between proteins and chromogen on a tooth surface.

In certain embodiments, hydrogen-bond disrupting agents change the tertiary structure of peptide molecules and denature proteins.

In certain embodiments, hydrogen-bond disrupting agents can include, but are not limited to, urea, arginine, guanidine, thiourea, allantoin, and combinations thereof.

In certain embodiments, the stain prevention compounds can include tannase, plasdone, peroxydone, subtilisin, sodium lauryl sulfate, sodium hexametaphosphate and combinations thereof.

The stain prevention formulations of the presently disclosed subject matter can be used in various consumer products, including food products, confectionary products, oral products, pet products (i.e., chew sticks) dentifrice, toothpaste, mouthwash, mouth spray, tobacco, and pharmaceuticals. The presently disclosed subject matter can be incorporated into oral products using conventional procedures and equipment and suitable additional components known in the art.

The present disclosure further provides stain prevention formulations in a confectionery product. In certain embodiments, the confectionery product is a chewy candy or a compressed mint. In other embodiments, the stain prevention compounds can be incorporated into chewing gum formulations.

### 3. Stain Prevention Chewing Gums

The presently disclosed subject matter can be incorporated into chewing gum using conventional procedures and equipment and suitable additional components known in the art.

A chewing gum product contains a water-insoluble chewable gum base portion, a water-soluble bulk portion and other ingredients such as flavors, sensates, and high potency sweeteners. The water-soluble portion can dissipate with a portion of the flavor over a period of time during chewing. The gum base portion is retained in the mouth throughout the chew.

In certain embodiments, stain prevention compounds can be mixed and added to the water-soluble portion bulk portion. In other embodiments, stain prevention compounds can be mixed and added to the water-insoluble base portion.

In certain embodiments, the insoluble gum base comprises elastomers, elastomer solvents, plasticizers, waxes, emulsifiers and/or inorganic fillers. In certain embodiments, the insoluble gum base can comprise elastomers, elastomer solvents, plasticizers, waxes, emulsifiers and/or inorganic fillers. Plastic polymers, such as polyvinyl acetate, which can behave as plasticizers, can also be included. In certain embodiments, plastic polymers can include but are not limited to, polyvinyl laureate, polyvinyl alcohol and polyvinyl pyrrolidone. Non-limiting examples of elastomers can include polyisobutylene, butyl rubber, (isobutylene-isoprene copolymer) and styrene butadiene rubber, as well as natural masticating substances such as chicle, etc. In certain embodiments, elastomer solvents can include resins such as terpene resins. In certain embodiments, the plasticizers are fats and oils, including but not limited to, tallow, hydrogenated and partially hydrogenated vegetable oils, and cocoa butter. In certain embodiments, the waxes include, but are not limited to, paraffin, microcrystalline and natural waxes such as beeswax and carnauba.

In certain embodiments, the chewing gum further contains one or more flavor components that are derived from artificial or natural sources or combinations thereof. In certain embodiments, the chewing gum can contain sugar, or may be sugar-free. In certain embodiments, the chewing gum can comprise high potency sweeteners including, but not limited to, synthetic substances, saccharin, thaumatin, alitame, saccharin salts, aspartame, sucralose, stevia, and acesulfame.

In certain embodiments, the insoluble gum base constitutes from about 5% to about 95% by weight of the gum. In certain embodiments, the insoluble gum base comprises from about 10% and about 50% by weight of the gum or from about 20% to about 35% by weight of the gum.

In certain embodiments, the high potency sweetener can comprise from about 0.02% to about 1.0%, or from about 0.05% to about 0.5% by weight of the chewing gum formulation.

In certain embodiments, the chewing gum has a neutral pH, e.g., from about 5.9 to about 8.0. In other embodiments, the chewing gum has a pH from about 7.5 to about 10.5.

In certain embodiments, the chewing gum formation process can include modification of one or more ingredients by encapsulation. In certain embodiments, encapsulation modifies the release of, for example, stain prevention compounds, from the chewing gum by modifying the solubility or dissolution rate. Any standard technique which gives partial or full encapsulation of the stain prevention agents can be used. In certain embodiments of the presently disclosed subject matter, encapsulation techniques include, but are not limited to, extrusion, spray drying, spray chilling, fluid-bed coating, and coacervation.

In certain embodiments, suitable encapsulating materials can include, but are not limited to, water-soluble sugar or sugar alcohol such as sorbitol, isomalt, dextrose, erythritol, lactitol, maltitol, mannitol, xylitol, hydrogenated corn syrup and mixtures thereof. In certain embodiments, encapsulating materials can also include hydrocolloids such as water soluble starch, modified starch, hydroxyl methyl cellulose, hydroxypropyl methylcellulose (HPMC), sodium alginate; cyclodextrins such as alpha, beta and gamma cyclodextrins; other polymers such as polyvinyl acetate (PVAC) and polyvinylpyrrolidone (PVP) and combinations thereof. In certain embodiments, the coating compositions can be susceptible to water permeation to various degrees. In certain embodiments, the coating composition is a food grade material.

The chewing gum can include one or more of the following: anti-microbial agents; anti-plaque agents; physiological cooling agents; breath-freshening agents; breath-freshening and mouth odor masking flavors; dental active agents; and combinations thereof.

Dental active agents include but are not limited to fluoride, anti-inflammation compounds, anti-cavity compounds, calcium salts, phosphate salts and mixtures thereof.

Anti-microbial and anti-plaque agents include, but are not limited to, cardamom oil, magnolia bark extract (MBE), cranberry, geraniol, cinnamaldehyde, peppermint, triclosan, chlorhexidine, cetyl pyridinium chloride (CPC), LAE and mixtures thereof. In certain embodiments, magnolia bark extract is present in the gum product in an amount of about 0.01 to about 5% by weight of the chewing gum product.

Physiological cooling agents include menthol N-2,3-trimethyl-2-isopropyl butanamide, 3-1-menthoxypropane-1,2-diol, N-ethyl-p-menthane-3-carboxamide, menthane ketals, menthyl succinate, isopulegol, menthyl glutarate, and mixtures thereof.

Breath-freshening agents include but are not limited polyphenols, mushroom extracts, allyl isothiocyanate (AITC), magnolia bark extract, other antibacterial agents that kill germ causing bad breath, or mixtures thereof. In certain embodiments, AITC is present in the gum product in an amount of about 0.01 to about 1% by weight of the chewing gum product.

Breath-freshening and mouth odor masking flavors include but are not limited to cinnamon, mint, wintergreen, fruit flavors and mixtures thereof.

In certain embodiments, the chewing gum formulation can include one or more stain prevention compounds.

The present application relates to stain prevention formulations that include at least one, two, three or more stain prevention compounds. The formulations can be incorporated into chewing gum formulations as discussed above while providing a mild taste and without damaging a consumer's teeth.

In certain embodiments, the stain prevention formulation comprises at least one, two, three or more stain prevention compounds selected from the group consisting of lauroyl arginine ethyl ether (LAE), sodium tripolyphosphate (STPP or STP), sodium pyrophosphate (SPP), sodium metatriphosphate (SMTP), urea, arginine, guanidine, thiourea, allantoin, carboxamidines and combinations thereof.

In certain embodiments, the stain prevention formulation comprises at least one, two, three or more encapsulated stain prevention compounds selected from the group consisting of lauroyl arginine ethyl ether (LAE), sodium tripolyphosphate (STPP or STP), sodium pyrophosphate (SPP), sodium metatriphosphate (SMTP), urea, arginine, guanidine, thiourea, allantoin, and combinations thereof. In certain embodiments, the stain prevention chewing gum formulation comprises from about 0.01% to about 5% encapsulated at least one, two, three or more stain prevention compounds. In certain embodiments, the stain prevention chewing gum formulation comprises about 2% encapsulated at least one, two, three or more stain prevention compounds. In certain embodiments, the stain prevention chewing gum formulation comprises about 2% encapsulated urea. In certain embodiments, the stain prevention chewing gum formulation comprises about 2% encapsulated STP.

In certain embodiments, the stain prevention formulation includes at least two stain prevention compounds. In certain embodiments, the two stain prevention compounds include one surface modifying agent and one hydrogen-bond disrupting agent.

In certain embodiments, the stain prevention formulation includes a combination of STP and urea, STP and LAE, SPP and LAE, or SPP and urea. In certain embodiments the stain prevention formulation includes about 0.1% STP and 0.1%, 0.25%, 0.5% or 1% urea. In certain embodiments the stain prevention formulation includes about 0.1% STP and 0.01%, 0.025%, 0.05% or 0.1% LAE. In certain embodiments the stain prevention formulation includes about 0.1% SPP and 0.025% or 0.05% LAE. In certain embodiments the stain prevention formulation includes about 0.1% SPP and 0.5% or 1% urea.

In an alternative embodiment, the chewing gum contains 0.01% to about 0.5% lauroyl arginine ethyl ether and 0.01% to about 5% sodium tripolyphosphate by weight of the chewing gum product. In yet another embodiment, the chewing gum contains 0.1% to about 0.2% lauroyl arginine ethyl ether and 1.0% to about 2.0% sodium tripolyphosphate by weight of the chewing gum.

In certain embodiments, the chewing gum comprises from about 0.01 to about 10% of a stain prevention formulation. In certain embodiments, the chewing gum comprises from about 0.2 to about 5% of a stain prevention formulation.

### 4. Stain Prevention Chewy Candies

In certain embodiments, breath-freshening formulations of the presently disclosed subject matter can be incorporated into confectionery products including, but not limited to, cakes, cookies, pies, candies (hard and soft), compressed mints, chewing gums, gelatins, ice creams, sorbets, jams, jellies, chocolates, fudge, fondant, liquorice, taffy, and combinations thereof. Oral products include, but are not limited to dentifrice, toothpaste, mouthwash, mouth spray, tobacco, and pharmaceuticals.

As embodied herein, the breath-freshening formulations are preferably incorporated in a chewy confectionery product. The oral products of the presently disclosed subject matter are used in a chewy confectionery product. Non-limiting examples of chewy confections include jellies, gummies, caramels, nougats, and taffies. The confections include a variety of shapes, flavors, textures, and sizes. Non-limiting examples of jelly candy products include jellybeans, gummy bears, and licorice sticks. Caramels are emulsions of fat droplets in a syrup made of sugars and hydrated milk proteins. Nougats are non-grained, firm, chewy confections having a light aerated texture. Taffies are also aerated, usually by a pulling machine, to produce a confection with a high degree of chewiness.

In general, a candy product typically comprises a mixture of base materials, thickeners, colorants and flavors. The base material may be a sugar or a polyol. Among the sugars that may be used are sucrose, dextrose, lactose, maltose and other common sugars. In addition, base materials may include non-sugar bulking agents. Among these are polyols such as sorbitol, maltitol, mannitol, xylitol, hydrogenated isomaltulose, lactitol, erythritol and combinations thereof. High intensity sweeteners such as acesulfame K, aspartame, alitame, sucralose, glycyrrhizin, saccharin and cyclamates may also be included with the base materials. The candy product may also include a sugarless sweetener. Sugarless sweeteners include components with sweetening characteristics but which are devoid of the commonly known sugars and comprise, but are not limited to, sugar alcohols such as sorbitol, mannitol, xylitol, hydrogenated starch hydrolyzates, maltitol and the like, alone or in any combination.

For the candy product to give a long chew, it should include a material that would create a candy product that is tough, elastic, chewy, and of reduced solubility in water. Such materials include pectin, gelatin, cellulose gum, colloid gum (such as xanthan, carageneenan, or locust bean), substituted or crosslinked starches and/or other such long chain materials.

Thickeners include corn syrup, gelatin, pectin, and other common thickeners and are added in amounts that achieve the desired organoleptic effect. In one embodiment of the present invention, the candy product is a chewy candy product. The chewy candy product can include sugar, corn syrup, fat and optionally, gelatin, which provide the desired chewy texture. Preferably, the chewy candy product comprises gelatins and fats in the necessary amounts to achieve the target texture of the candy product.

In another embodiment of the present invention, the candy product is a gummy candy product. The gummy candy product can include sugar, corn syrup, gelatin and optionally, pectin, which provide the desired gummy texture. In an alternate embodiment of the present invention, the candy product comprises a soft chewy or a soft gummy candy product. The soft chewy gummy candy product includes sugar, corn syrup and pectin, with pectin to achieve the desired soft chewy gummy texture. In yet another alternate embodiment, the candy product comprises a pressed chewy product. The pressed chewy candy product can include sugar, corn syrup and fat, which provide the desired chewy texture.

In certain embodiments, the chewy candy has a pH from about 5.9 to about 7.5, or from about 7.5 to about 10.5, or from about 10.5 to 12.0.

Pressed chewy candy products can also include binders and lubricants. Binders that are commonly used are natural gums and hydrocolloids such as gum arabic, guar gum, agar, alginates, gum tragacanth, gelatin, corn syrups, modified starches, maltodextrins and optionally agglomerated dextrose. Most commonly used binders are gelatin, gum arabic or corn syrups. When non-sugar polyols such as sorbitol are used as the base material, binders are not needed for binding since many of these polyols are easily compressed to form candy products. In some cases polyols such as sorbitol can also act as a binder and may be combined with sugar to form the base materials for the compressed chewy candy product.

Lubricants may be used to give good release from the press tooling or die and punches. A variety of lubricants or non-stick agents may be used in a pressed chewy candy product to act as release agents. Some of these are starch, acetylated monoglycerides, waxes, lecithins, emulsifiers, and mono-, di-, tristearates. The most common of these lubricants are magnesium or calcium stearate and stearic acid. Solid lubricants may be added to the candy product to help form the candy product and allow for its release. In some instances, low levels of flow agents such as silicon dioxide are added to the pressed chewy candy product composition to help the flow of the mixture into the press tooling.

Colors and other additives are also contemplated for use in the candy products of the presently disclosed subject matter.

In a preferred embodiment, LAE and STP is used in combination in a sugar-free chewy confection application. The stain prevention formulation can include a combination of STP and urea, STP and LAE, SPP and LAE, or SPP and urea. In certain embodiments the stain prevention formulation includes about 0.1% STP and 0.1%, 0.25%, 0.5% or 1% urea. In another embodiment the stain prevention formulation includes about 0.1% STP and 0.01%, 0.025%, 0.05% or 0.1% LAE. In certain embodiments the stain prevention formulation includes about 0.1% SPP and 0.025% or 0.05% LAE.

In certain embodiments the stain prevention formulation includes about 0.1% SPP and 0.5% or 1% urea. In an alternative embodiment, the STP and LAE may be added to the chewy candy neat or in an encapsulation matrix, alone or in combination. In an alternative embodiment, the chewy candy contains 0.01% to about 0.5% lauroyl arginine ethyl ether and 0.01% to about 5% sodium tripolyphosphate by weight of the chewy candy product. In yet another embodiment, the chewy candy contains 0.1% to about 0.2% lauroyl arginine ethyl ether and 1.0% to about 2.0% sodium tripolyphosphate by weight of the chewy candy.

### 5. Stain Prevention Compressed Mints

The presently disclosed subject matter can be incorporated into mint confectionaries, and more particularly into compressed mint products using conventional tablet pressing procedures and equipment and suitable additional components known in the art. Compressed mints of the presently disclosed subject matter can contain sugar or can be sugarfree. In addition to the disclosed cooling compositions, other suitable flavoring agents can be included as well as ingredients that give a tingling sensation. In the case of products with multiple layers, each layer may have different flavoring agents or levels. In one embodiment, the compressed mint can comprise a coating layer covering at least a portion of the product. In that case, the coating layer can contain flavoring agents at a level higher than any flavoring agents in the remainder of the product.

The compressed mints can include one or more of the following: anti-microbial agents; physiological cooling agents; breath-freshening agents; breath-freshening and mouth odor masking flavors; dental active agents; and combinations thereof.

In certain embodiments, the compressed mint has a pH from about 5.9 to about 7.5, or from about 7.5 to about 10.5, or from about 10.5 to 12.0.

In certain embodiments, the compressed mint tablet further contains one or more additional compounds including but not limited to sorbitol (e.g., 90-99%), acesulfame K (e.g., 0.05-0.5%), sucralose (e.g., 0.05-0.5%), magnesium stearate (e.g., 0.5-2%), Neobee oil (e.g., 1-2%), peppermint oil (e.g., 0.5-2%), and combinations thereof by weight of the compressed mint product.

In a preferred embodiment, LAE and STP is used in combination in a sugar-free compressed mint application. The stain prevention formulation can include a combination of STP and urea, STP and LAE, SPP and LAE, or SPP and urea. In certain embodiments the stain prevention formulation includes about 0.1% STP and 0.1%, 0.25%, 0.5% or 1% urea.

In another embodiment the stain prevention formulation includes about 0.1% STP and 0.01%, 0.025%, 0.05% or 0.1% LAE. In certain embodiments the stain prevention formulation includes about 0.1% SPP and 0.025% or 0.05% LAE. In certain embodiments the stain prevention formulation includes about 0.1% SPP and 0.5% or 1% urea. In an alternative embodiment, the STP and LAE may be added to the compressed mint neat or in an encapsulation matrix, alone or in combination.

In an alternative embodiment, the compressed mint contains 0.01% to about 0.5% lauroyl arginine ethyl ether and 0.01% to about 5% sodium tripolyphosphate by weight of the compressed mint product. In yet another embodiment, the compressed mint contains 0.1% to about 0.2% lauroyl arginine ethyl ether and 1.0% to about 2.0% sodium tripolyphosphate by weight of the compressed mint product.

### EXAMPLES

The presently disclosed subject matter will be better understood by reference to the following Examples, which are provided as exemplary of the disclosed subject matter, and not by way of limitation.

### Example 1: Evaluation of Stain Prevention Chewing Gum

In this Example, the stain prevention effect of chewing gum comprising stain prevention compounds was evaluated by a disc stain assay, a bovine teeth test, and a release test.

### A. Methods

I. In Vitro Hydroxyapatite Disc (HAD) and Bovine Teeth Tests

Urea, STP, SPP and SMTP (all food grade) were purchased from Spectrum Chemicals. LAE P/100 was received from Vedeqsa Inc.

HAD assay and the bovine teeth test were employed by methods generally known in the art for screening stain prevention actives. HAD assays generally included pre-coating a HAD disc with saliva for 1-2 hours and subsequently exposing the disc to a staining broth containing tea, coffee, gastric mucin, trypticase soy broth and saliva for four days. Each day, the disc was moved from staining broth to a solution with a stain prevention formulation three times. The process was repeated for four days. The disc was dried and the color was measured. The bovine teeth test generally included sanding, grinding and polishing bovine teeth, assembling a tooth block, coating the teeth with saliva, and staining and treating the tooth as described for the HAD assay. The color of the bovine teeth was measured before and after staining.

Stain prevention solutions were prepared as summarized in Tables 1 and 2.

TABLE 1
Stain prevention solutions.

| Stain Preventing Compound | % by weight |
|---|---|
| SPP | 0.25; 0.5 |
| STP | 0.1; 0.2 |
| Hydrogen Peroxide | 0.25 |
| Urea | 0.1; 0.25; 0.5; 1; 3; 6 |
| Arginine | 0.25; 0.5; 1 |
| LAE | 0.025; 0.05 |

TABLE 2
Combinations of Stain Preventing Compounds

| | SPP | STP | urea | LAE |
|---|---|---|---|---|
| | (% by weight) | (% by weight) | (% by weight) | (% by weight) |
| 2A | — | 0.1 | 0.1 | — |
| 2B | — | 0.1 | 0.25 | — |
| 2C | — | 0.1 | 0.5 | — |
| 2D | — | 0.1 | 1 | — |
| 2E | — | 0.1 | — | 0.01 |
| 2F | — | 0.1 | — | 0.025 |
| 2G | — | 0.1 | — | 0.05 |
| 2H | — | 0.1 | — | 0.1 |
| 2I | 0.1 | — | — | 0.025 |
| 2J | 0.1 | — | — | 0.05 |
| 2K | 0.1 | — | 0.5 | — |
| 2L | 0.1 | — | 1 | — |

To evaluate stain prevention effect, the whitening index of the teeth before and after staining and treatment was calculated. The white index, W, is defined as:

W*=\sqrt{(L*−1.00)²+(a*)²+(b*)²}

And the change of

ΔW*=W_{treated}*−W_{control}*

A pure white substance will have W value of 0, and a pure black with W of 100. The higher the value of ΔW, the whiter the sample would be.

In total, 4-6 HA discs or bovine teeth were tested for each compound or combination. The values were averaged. No-treatment was used as a negative control, and hydrogen peroxide (0.25%, which was demonstrated to be efficacious for stain prevention) was used as a positive control.

II. Preparation of Chewing Gum and Chewy Candy Formulations

### Chewing Gum

Chewing gum formulations were prepared by standard methods with stain prevention compounds including STP, SPP, urea and LAE. This included pellet, stick and tab forms. A peppermint gum formulation was prepared in pellet form with LAE in the center layer.

Preparation of the chewing gum include heating gumbase 70° C. Polyols, high intense sweeteners, flavors and stain-prevention active compounds were then added to the mixer. The gum was sheeted and cut to pieces. For the coated (pellet) gum, the gum center was loaded in a coating pan and further coated by a coating syrup containing polyol, high intense sweeteners, flavors and emulsifier. Table 3 describes various chewing gum formats (pellet, stick, and tab) tested.

### Chewy Candy

Lycasin and mannitol were heated to 135° C. and mixed well. The mixture was cooled to 90° C. STP and LAE were added and mixed well to Chewy Candy A and B. For Chewy Candy A, the jelly mixture as depicted in Table 6 was added with the STP and LAE. Next, peppermint oil, palm oil, distilled monoglyceride and lecithin were added to the mixture and stirred to mix well. Separately, gelatin and deionized (DI) water were heated to 90° C. and mixed well, and added to the lycasin mixture. The product were further stirred and cooled until a consistent soft chewy candy blend was formed. The chewy blend was sheeted and cut into 3.0 g piece weight.

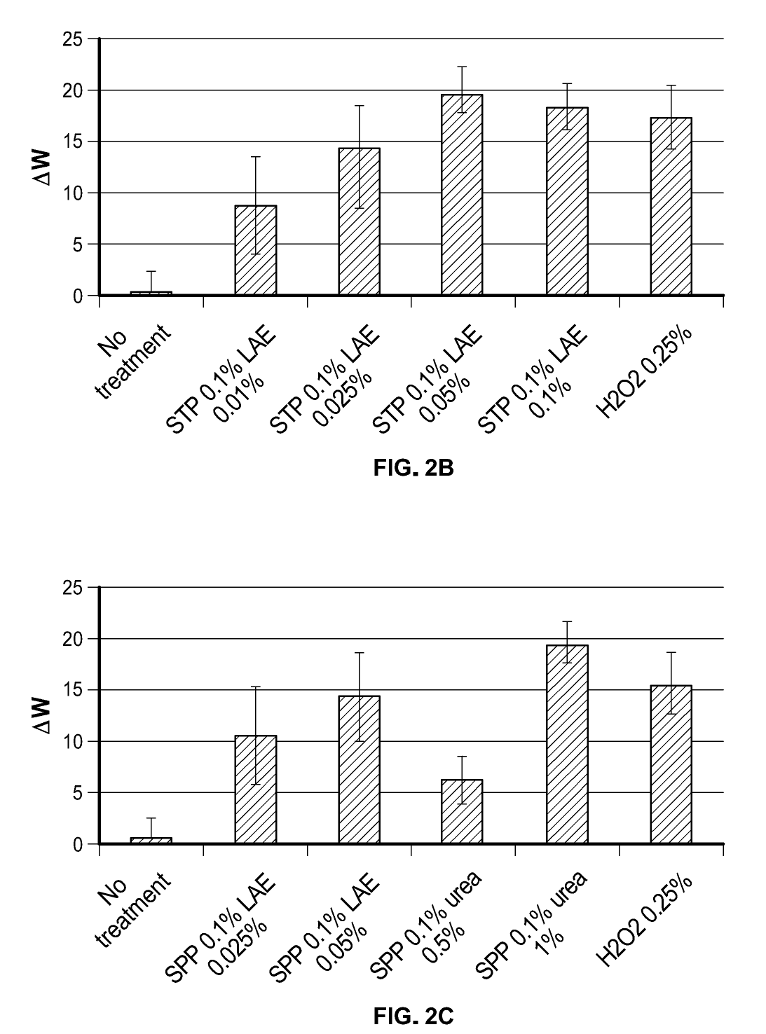

Table 3 shows the formulations of three different forms of peppermint chewing gum containing LAE. FIG. 3 shows the release of LAE from these three different forms of gum matrix.

TABLE 3
Chewing gum formulas and formats

| | Pellet gum | Stick gum | Tab gum |
|---|---|---|---|
| Ingredient | 1.5 g | 2.7 g | 2.0 g |
| Sorbitol | 42.3% | 70.4% | 57.0% |
| Gum base | 31.0% | 26.0% | 28.0% |
| Calcium carbonate | 14.0% | — | — |
| Xylitol | 5.20% | — | — |
| Glycerin | 3.4% | 1.0% | 10.0% |
| Peppermint oil | 1.1% | 2.0% | 2.2% |
| Menthol | 0.70% | 0.2% | 0.5% |
| STP | 2.0% | — | 2.0% |
| LAE P100 | 0.1% | 0.2% | 0.1% |
| High potency | 0.2% | 0.2% | 0.2% |
| sweetener | | | |
| TOTAL | 100.0% | 1000.% | 100.0% |

A release of LAE from 13%-25% was observed by high performance liquid chromatography (HPLC) for a 20-minute chew in each type of chewing gum form tested. This release delivered up to 1.5 mg or 100 ppm of LAE to oral cavity. Release of STP and urea were also analyzed and were found between 85%-100% after a 20-minute chew.

TABLE 4
Stain-prevention gum formulas in stick form.

| | | Stain prevention | Stain prevention |
|---|---|---|---|
| Compound | Control gum | gum A | gum B |
| Gum Base | 28.0% | 28.0% | 28.0% |
| Sorbitol | 59.1% | 57.1% | 55.1% |
| LAE P100 | — | — | 0.1% |
| STP | — | 2.0% | 2.0% |
| Urea | — | 2.0% | — |
| Glycerol | 10.0% | 10.0% | 10.0% |
| Peppermint oil | 2.0% | 2.0% | 2.0% |
| Cooling agent | 0.18% | 0.18% | 0.18% |
| Menthol | 0.5% | 0.5% | 0.5% |
| High potency | 0.21% | 0.21% | 0.21% |
| sweetener | | | |
| TOTAL | 100% | 100% | 100% |

In an alternative embodiment, before incorporating into the gum, urea and STP are encapsulated by a polyvinyl acetate (PVAC) matrix. Each active compound was first dissolved and spray-dried to obtain urea or STP particles. The particles were then extruded with medium molecular weight PVAC, and particulated to the desired particle size. The final loading of urea or STP was 15% by weight of the encapsulation matrix.

Sugarfree gums containing 2% encapsulated urea and 2% encapsulated STP were chewed for 0.5, 1, 2, 5 and 12 minutes by 3-6 sensory trained panelists. All gum cuds were collected after chewing and analyzed. Encapsulated gums were tested together with gums containing 2% neat urea, and 2% neat STP. Sensory testing revealed a noticeable improvement of bitterness taste after encapsulation of urea.

TABLE 5
Sugar-free chewy candy formulas

| | Control chewy | Chewy candy A | Chewy candy B |
|---|---|---|---|
| Ingredient | candy | (low gel) | (high gel) |
| Lycasin | 73.0% | 72.0% | 72.3% |
| Mannitol | 15.7% | 14.38% | 14.38% |
| Peppermint oil | 0.5% | 0.5% | 0.5% |
| Gelatin | 2.5% | — | 2.5% |
| STP | — | 2.0% | 2.0% |
| LAE P100 | — | 0.02% | 0.02% |
| DI water | 3.9% | — | 3.9% |
| Palm oil | 4.0% | 4.0% | 4.0% |
| Distilled | 0.2% | 0.2% | 0.2% |
| monogylceride | | | |
| Lecithin | 0.2% | 0.2% | 0.2% |
| Jelly mixture | — | 6.7% | |
| TOTAL | 100.0% | 100.0% | 100.0% |

TABLE 6
Jelly mixture for low gel chewy candy.

| Ingredient | Concentration % |
|---|---|
| Dextrin | 10.8% |
| Waxy Maize Starch | 4.8% |
| Gelatin | 9.0% |
| Mannitol | 16.3% |
| Lycasin | 18.1% |
| D.I. Water | 41.0% |
| Subtotal | 100.0% |

### B. Results and Discussion

I. In vitro (HAD) and bovine teeth tests

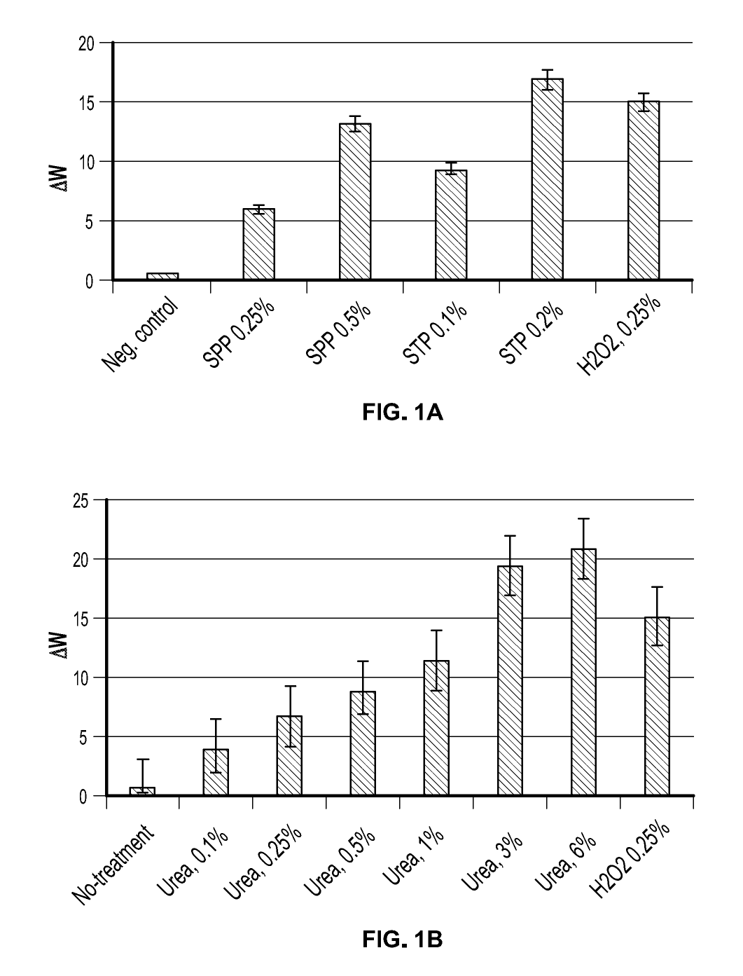

FIG. 1 shows the dose response of SPP, STP, Urea and LAE as summarized in Table 1, for stain-prevention effect by bovine teeth tests. SPP, STP, Urea and LAE were very effective compounds for stain-prevention. Compared with SPP, STP was more effective for the same concentration level.

It has been reported that 50% of population will visually observe a color difference for a change of ΔE (or ΔW)>=2.7. 99% of population will observed a difference for ΔE (or ΔW)>=3.7. Taking into consideration other factors such as tooth translucence, lighting condition, etc., a majority of population will observe a color difference with ΔE (or ΔW)>=7.5. (See, Paravina R D, et al. Color Difference Thresholds in Dentistry, J. Esthetic and Restorative Dentistry, 2015; 27: S1-S9; Chu S J, Trushkowsky R D, Paravina R D. Dental color matching instruments and systems, review of clinical and research aspects. 2010; 38s: e2-e16. which are hereby incorporated by reference.) This example found that compounds including STP (0.1% or above), SPP (0.5% and above), urea (0.5% and above), LAE (0.05% and above) and hydrogen peroxide (0.25% and above) show a significant visible whitening effect.

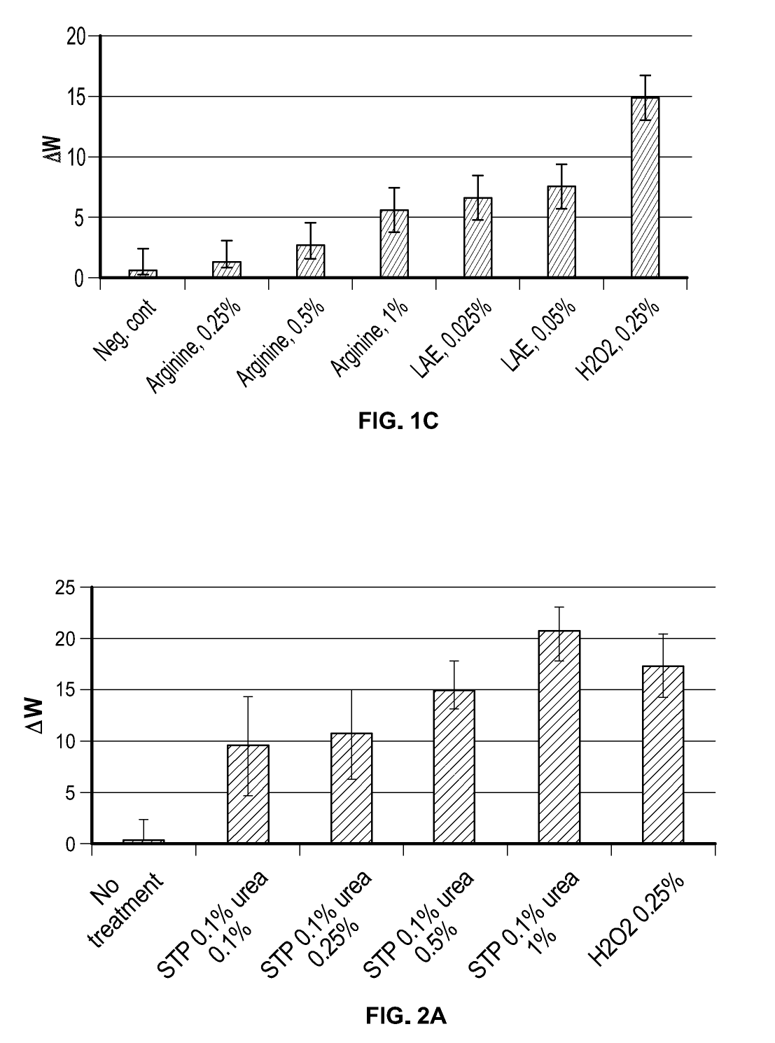

FIG. 2 shows the combinations of STP-urea, STP-LAE, SPP-LAE, and SPP-urea as summarized in Table 2. The combinations showed significant enhancement and/or synergistic effect on stain prevention, i.e., one can reduce both concentration of STP, LAE or urea to achieve the same or a better effect on stain-prevention. Among the binary combinations, STP-urea and STP-LAE showed to be the most effective. SPP-urea, SPP-LAE and LAE-urea were not as effective as the STP combos. The binary combinations were also more effective than a tri-blend of STP, urea and LAE.

### Example 2: Chewing Machine Study

In this Example, chewing gum and chewy candy was tested with an in vitro chewing machine to assess the prevention effect of new tooth stain formation.

### A. Materials and Methods

Two stain prevention gums were formulated. Gum A comprised a combination of STP/urea. Gum B comprised a combination of neat STP/LAE. A mint gum tab was used as a control. These formulas are depicted in Table 3 above. In addition, two stain prevention chewy candies were formulated. Chewy Candy A and Chewy Candy B comprised a combination of STP and LAE. Chewy Candy A contained a “low gel” and Chewy Candy B contained a “high gel.”

For the chewing machine test, a mechanical instrument, which was developed by Kleber et al. to simulate the human mastication of chewing gum was employed. (See, Kleber C J, Schimmele R G, Putt M S, Muhler J C: A mastication device designed for the evaluation of chewing gums. J. Dent. Res. 1981; 60: 109-114, hereby incorporated by reference.) Before the treatment, the baseline L*a*b* stain scores of the enamel specimens were determined. The tooth block was attached to a staining apparatus that was designed to provide alternate immersion into the staining broth and air-drying of the specimens. The apparatus was placed in an incubator at 37° C. for 24 hrs. The Teflon rod that carried tooth blocks in the apparatus was rotated constantly at 1.5 rpm. The staining broth contained coffee, tea, gastric mucin, trypticase soy broth and M. luteus culture to stimulate stain formation. During the treatment, tooth block was loaded to a chewing machine. 15 ml of modified artificial human saliva was place in the reservoir. One piece of gum was placed between the repositioning paddles directly over the lower tooth specimens. The mastication motor was started and the gum was chewed for 10 min each time, and 3 times each day.

After running for each chewing, bovine teeth blocks were placed back in to the staining apparatus. The process was repeated for 14 days. The color of teeth was measured at t=0, 7 days and 14 days. Each test was repeated 4 times.

### B. Results

The color measurements of the teeth at t=0, 7 days and 14 days is summarized in Tables 7-9.

TABLE 7
Baseline Of Teeth Before Staining And Treatment

| | Spectrophotometer Readings | | |
|---|---|---|---|
| | (mean score ± standard deviation; n = 4) | | |
| Sample | L* | a* | b* |
| Gum A | 71.37 ± 0.99 | 0.49 ± 0.29 | 4.85 ± 1.00 |
| Gum B | 71.65 ± 0.70 | 0.53 ± 0.37 | 5.09 ± 1.71 |
| Gum Control | 71.64 ± 1.61 | 0.48 ± 0.60 | 5.43 ± 1.28 |
| No treatment | 71.83 ± 1.62 | 0.63 ± 0.51 | 5.50 ± 0.90 |
| Candy A | 69.83 ± 1.95 | 0.87 ± 0.87 | 4.62 ± 2.12 |
| Candy B | 69.61 ± 1.43 | 0.76 ± 0.69 | 4.35 ± 0.65 |
| Candy Control (no | 69.77 ± 2.56 | 1.02 ± 0.55 | 4.59 ± 3.96 |
| actives) | | | |
| Tab 2% STP/0.1% LAE | 69.45 ± 2.20 | 0.56 ± 0.28 | 4.44 ± 1.11 |
| Pellet 2% STP/0.1% | 69.34 ± 2.90 | 0.67 ± 0.64 | 4.65 ± 2.37 |
| LAE | | | |

TABLE 8
Extrinsic Stain On Teeth After 7 Days Of Treatments

| | Spectrophotometer Readings | | |
|---|---|---|---|
| | (mean score ± standard deviation; n = 4) | | |
| Sample | L* | a* | b* |
| Gum A | 60.15 ± 1.57 | 3.64 ± 0.74 | 13.90 ± 1.87 |
| Gum B | 62.69 ± 0.56 | 3.12 ± 0.38 | 13.46 ± 2.84 |
| Gum Control | 57.86 ± 1.92 | 4.47 ± 0.62 | 17.46 ± 0.92 |
| No treatment | 55.76 ± 0.32 | 6.75 ± 0.40 | 21.37 ± 0.64 |
| Candy A | 55.97 ± 1.89 | 5.75 ± 1.35 | 18.85 ± 2.22 |
| Candy B | 55.50 ± 1.09 | 5.99 ± 0.76 | 18.69 ± 2.01 |
| Candy Control (no | 49.89 ± 2.84 | 8.29 ± 1.84 | 22.89 ± 2.56 |
| actives) | | | |
| Tab 2% STP/0.1% LAE | 56.54 ± 1.53 | 5.55 ± 0.61 | 19.17 ± 0.65 |
| Pellet 2% STP/0.1% | 56.73 ± 1.86 | 5.72 ± 1.02 | 18.80 ± 3.33 |
| LAE | | | |

TABLE 9
Extrinsic Stain On Teeth After 14 Days Of Treatments

| | Spectrophotometer Readings | | |
|---|---|---|---|
| | (mean score ± standard deviation; n = 4) | | |
| Gum | L* | a* | b* |
| Gum A | 55.97 ± 2.34 | 4.72 ± 2.14 | 12.47 ± 1.26 |
| Gum B | 59.08 ± 1.24 | 3.13 ± 0.33 | 12.28 ± 2.08 |
| Gum Control | 51.57 ± 0.92 | 5.49 ± 0.67 | 16.43 ± 0.43 |
| No treatment | 47.86 ± 0.61 | 8.93 ± 0.50 | 21.45 ± 0.30 |
| Candy A | 53.62 ± 2.46 | 5.37 ± 1.09 | 20.24 ± 2.84 |
| Candy B | 53.36 ± 1.11 | 6.36 ± 0.70 | 20.38 ± 1.65 |
| Candy Control (no | 45.59 ± 3.70 | 10.09 ± 1.85 | 22.75 ± 2.22 |
| actives) | | | |
| Tab 2% STP/0.1% LAE | 52.27 ± 2.04 | 5.85 ± 0.31 | 17.54 ± 0.74 |
| Pellet 2% STP/0.1% | 55.68 ± 1.88 | 5.72 ± 1.23 | 18.78 ± 3.41 |
| LAE | | | |

The overall change in color of the teeth at 7 days and 14 days is summarized in Tables 10-11.

TABLE 10
Change In Extrinsic Stain On Teeth After 7 Days Of Treatments

| | Spectrophotometer Readings | | | |
|---|---|---|---|---|
| | (mean score ± standard deviation; n = 4) | | | |
| Sample | ΔL* | Δa* | Δb* | ΔE |
| Gum A | −11.22 ± 1.58 | 3.15 ± 0.68 | 9.06 ± 1.02 | 14.79 ± 1.64 |
| Gum B | −8.96 ± 0.74 | 2.59 ± 0.61 | 8.38 ± 1.43 | 12.57 ± 1.28 |
| Gum Control | −13.78 ± 1.03 | 4.00 ± 0.70 | 12.03 ± 0.77 | 18.76 ± 0.45 |
| No treatment | −16.07 ± 1.38 | 6.12 ± 0.30 | 15.88 ± 1.28 | 23.42 ± 1.56 |
| Candy A | −13.85 ± 1.26 | 4.87 ± 1.00 | 14.22 ± 0.39 | 20.47 ± 1.27 |
| Candy B | −14.10 ± 1.98 | 5.23 ± 1.30 | 14.34 ± 1.40 | 20.80 ± 2.61 |
| Candy Control (no | −19.88 ± 1.51 | 7.27 ± 1.94 | 18.29 ± 2.37 | 28.08 ± 2.08 |
| actives) | | | | |
| Tab 2% STP/0.1% | −12.90 ± 1.31 | 4.99 ± 0.85 | 14.73 ± 0.83 | 20.24 ± 1.33 |
| LAE | | | | |
| Pellet 2% | −12.61 ± 1.31 | 5.05 ± 1.55 | 14.14 ± 2.27 | 19.64 ± 3.24 |
| STP/0.1% LAE | | | | |

TABLE 11
Change In Extrinsic Stain On Teeth After 14 Days Of Treatments

| | Spectrophotometer Readings | | | |
|---|---|---|---|---|
| | (mean score ± standard deviation; n = 4) | | | |
| Sample | ΔL* | Δa* | Δb* | ΔE |
| Gum A | −15.40 ± 1.39 | 4.22 ± 2.14 | 7.62 ± 1.56 | 17.82 ± 1.72 |
| Gum B | −12.57 ± 0.62 | 2.60 ± 0.58 | 7.20 ± 0.89 | 14.74 ± 0.74 |
| Gum Control | −20.07 ± 1.28 | 5.01 ± 0.84 | 11.00 ± 1.30 | 23.46 ± 1.39 |
| No treatment | −23.97 ± 1.20 | 8.30 ± 0.48 | 15.96 ± 1.17 | 29.30 ± 1.50 |
| Candy A | −16.21 ± 0.83 | 4.49 ± 0.82 | 15.61 ± 1.11 | 22.96 ± 1.17 |
| Candy B | −16.25 ± 1.64 | 5.60 ± 0.96 | 16.03 ± 1.14 | 23.52 ± 2.13 |
| Candy Control (no | −24.18 ± 2.31 | 9.07 ± 0.83 | 18.15 ± 1.85 | 31.66 ± 2.09 |
| actives) | | | | |
| Tab 2% STP/0.1% | −17.17 ± 0.38 | 5.29 ± 0.32 | 13.10 ± 0.49 | 22.24 ± 0.56 |
| LAE | | | | |
| Pellet 2% | −13.66 ± | 5.04 ± | 14.13 ± | 20.32 ± |
| STP/0.1% LAE | 3.02 | 1.29 | 2.86 | 4.36 |

The two treatment controls, Gum Control and Chewy Candy Control developed more intense stain than those groups that were treated with the respective test chewing gums and chewy candies. Furthermore, there were statistically significant separations of the prototype gums and chewy candies versus the control chewing gum and chewy candy, respectively.

The chewing gum results are also summarized in FIG. 5 which illustrates the ΔE for each gum at t=0 days, 7 days and 14 days. All gums showed a better ΔE than no gum. Gum A and Gum B showed significant stain prevention versus the control. Gum B was the best overall in reduction of stain formation. The chewy candy prototypes containing sodium tripolyphosphate (STP) and lauroyl arginine ether (LAE) in low gelatin and high gelatin formulas were comparable in their reduction of stain formation.

The inventors found that the pellet gums produced less stain than tab gums with the same actives. Without being held to any particular type of theory, this is most likely as a result of the mechanical action produced by the hard coating during the treatments.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the present disclosure is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications publications product descriptions, and protocols are cited throughout this application, the disclosures of which are incorporated herein by reference in their entireties for all purposes.

What is claimed is:

1. An oral product comprising at least one stain prevention compound selected from the group consisting of surface modifying agents, hydrogen-bond disrupting agents and combinations thereof.

2. The oral product of claim 1, wherein the oral product comprises at least two stain prevention compounds.

3. The oral product of claim 1, wherein the at least one surface modifying agent is selected from the group consisting of lauroyl arginine ethyl ether, sodium tripolyphosphate, sodium pyrophosphate, sodium metatriphosphate, and combinations thereof.

4. The oral product of claim 1, wherein the at least one hydrogen-bond disrupting agent is selected from the group consisting of urea, arginine, guanidine, thiourea, allantoin, and combinations thereof.

5. The oral product of claim 1, wherein the oral product comprises one surface modifying agent and one hydrogen-bond disrupting agent.

6. The oral product of claim 1, wherein the oral product is selected from the group consisting of chewing gum, chewy candy and compressed mint.

7. An oral product comprising at least two surface modifying agents selected from the group consisting of lauroyl arginine ethyl ether and sodium tripolyphosphate.

8. The oral product of claim 7 comprising:
a) 0.01% to about 0.5% lauroyl arginine ethyl ether; and,
b) 0.01% to about 5% sodium tripolyphosphate by weight of the oral product.

9. The oral product of claim 7 comprising:
a) 0.1% to about 0.2% lauroyl arginine ethyl ether; and,
b) 1.0% to about 2.0% sodium tripolyphosphate by weight of the oral product.

10. The oral product of claim 7, wherein at least one of the surface modifying agents is encapsulated.

11. The oral product of claim 7, wherein the oral product is a chewing gum product.

12. The oral product of claim 7 further comprising at least one hydrogen-bond disrupting agent selected from the group consisting of urea, arginine, and allantoin.

13. The oral product of claim 7 further comprising at least one hydrogen-bond disrupting agent is selected from the group consisting of guanidine and thiourea.

14. The oral product of claim 7, wherein the pH of the oral composition is from about 5.9 to about 12.

15. The oral product of claim 7, wherein the oral product is a chewy candy product.

16. A method of tooth staining prevention which comprises at least two surface modifying agents selected from the group consisting of lauroyl arginine ethyl ether, sodium tripolyphosphate, sodium pyrophosphate, sodium metatriphosphate, and combinations thereof into an oral product in the amount of about 0.01% to about 5.0% by weight of each surface modifying agent by weight of the oral product.

17. The method of claim 16, wherein the oral product is a coated chewing gum product comprising one surface modifying agent in the chewing gum center by admixing the ingredients until a uniform mixture is obtained, and thereafter incorporating the second surface modifying agent to the coating of the coated chewing gum product.

18. The method of claim 16, wherein the oral product further comprising at least one hydrogen-bond disrupting agent selected from the group consisting of urea.

19. The method of claim 17, wherein the coated chewing gum comprising at least one surface modifying agent by admixing in the chewing gum center is lauroyl arginine ethyl ether.
